(19) United States
(12) Patent Application Publication
(10) Pub. No.: US 2026/0256500 A1
(43) Pub. Date: Sep. 3, 2026

(54) Set Screw Retention Driver Assembly

(71) Applicant: K2M, Inc., Leesburg, VA (US)

(72) Inventors: Bryan D. Milz, Milford, NJ (US); Daniel Genovese, Great Falls, VA (US)

(73) Assignee: K2M, Inc., Leesburg, VA (US)

(21) Appl. No.: 19/553,973

(22) Filed: Mar. 2, 2026

Related U.S. Application Data
(60) Provisional application No. 63/764,613, filed on Feb. 28, 2025.

(51) Int. Cl. A61B 17/70

(52) U.S. Cl. CPC A61B 17/7082

(57) ABSTRACT

A system may include a set screw having a threaded portion and a cap. The system may further include a driver having a shank configured to engage the cap, and a retention tip including a flexible portion to retain the set screw to the driver. Upon tightening of the set screw, the cap is removed from the threaded portion and retained by the shank.

### CROSS-REFERENCE TO RELATED APPLICATIONS

This application claims the benefit of the filing date of United States Provisional Patent Application No. 63/764,613 filed February 28, 2025, the disclosure of which is hereby incorporated herein by reference in its entirety.

### BACKGROUND OF THE INVENTION

Spinal fusion surgery is performed to treat various spinal maladies, including cases of spinal deformity, spondylolisthesis, degenerative disc disease, and other conditions that benefit from spinal stabilizations. These procedures can take on many different forms, for instance, pedicle screws are screws designed for insertion into vertebral pedicles to provide a foundation for spinal fusion. After the screws are placed, spinal fixation rods are then inserted within coupling elements or tulip heads that are often polyaxially attached to the screw portions of the pedicle screws. This effectively creates a brace that prevents movement between the vertebrae and provides stability to the fusion site.

In ultimately fixing the spinal rods to the coupling elements, small lock screws (also known as set screws or locking caps), are typically used to secure the rods into place with the desired tension. This has the added benefit of also locking the coupling elements and screw portions in place with respect to one another. These set screws are often very small, as are the spaces into which they are placed. As such, placement of these set screws during a spinal fusion procedure is challenging and not without complications. Moreover, the problem may be exacerbated by the fact that such procedures often involve the use of several individual pedicle screws, and hence the need to place several set screws.

### SUMMARY OF THE INVENTION

A combination driver and set screw system is disclosed herein. The system permits the easy application of set screws to pedicle screws and other types of implant by providing a retained coupling between the two components and permitting the quick application of multiple set screws. This is particularly useful in the context of a spinal fusion procedure where multiple pedicle screws are implanted and ultimately locked to a spinal rod. The set screw includes a threaded portion and cap that are separable upon the application of a maximum torque. The driver includes a retention tip that acts to hold the set screw to the driver and a shank that provides a rotational force to the cap of the set screw. Upon reaching the maximum torque/force a weakened section joining the threaded portion and cap breaks, thereby permitting the two portions of the set screw to separate. The shank of the driver includes a length that permits the retention of separated set screw caps, which aids in speeding up the implantation process and prevents the inadvertent disposal of a separated cap within the body of a patient.

While discussed in connection with the application of set screws to pedicle screws during a spinal fusion procedure, the components disclosed herein may have applicability to other areas of surgery and even uses outside of the surgical arts. For instance, the set screws disclosed herein could be modified to exhibit more of a traditional screw configuration that could have applicability in the installation of bone plates and plates attached to spinal implants.

One aspect of the present disclosures is a system including a set screw having a threaded portion and a cap, and a driver having a shank configured to engage the cap and a retention tip including a flexible portion to retain the set screw to the driver. Upon tightening of the set screw, the cap is removed from the threaded portion and retained by the shank.

In other embodiments of the first aspect, the cap may be connected to the threaded portion by a weakened portion. The weakened portion may be designed to break upon the application of a maximum torque to the cap. The retention tip may be rotatable with respect to the shank and the retention tip may be connected to the shank by a screw. In other embodiments, the shank and cap may have cooperating surfaces, for instance, star shaped surfaces. The driver may include a handle, the shank being rotatable with respect to the handle. The driver may also be attached to a driving tool, such as, a drill.

Another aspect of the present disclosure is a method of attaching a set screw to an implant including the steps of engaging a retention tip a driver with a screw portion of the set screw, coupling a shaft of the driver with a cap of the set screw, imparting a force on the cap with the driver to screw the screw portion into the implant, and after fully screwing the screw portion into the implant, removing the cap from the set screw and retaining the cap on the shaft.

Other embodiments of this aspect may include imparting a maximum torque on the cap during the removing step. The method may also include sliding the cap along the shaft. The steps of the method may be conducted for multiple set screws and multiple caps may be retained on the shaft. The retention tip may include at least one flexible portion, and may include a plurality of flexible portions. The method may also include holding a handle of the driver while imparting the force. The imparting step may be conducted with a driving tool. The implant may be a pedicle screw.

Another aspect of the present disclosure is a system including a plurality of set screws each having a threaded portion and a cap, and a driver having a shank configured to engage the caps and a retention tip including a flexible portion to retain the set screws to the driver. Upon tightening of each set screw, the respective cap may be removed from the respective threaded portion and retained by the shank. The shank may include a length designed to retain a predetermined number of removed caps.

### BRIEF DESCRIPTION OF THE DRAWINGS

A more complete appreciation of the subject matter of the present disclosure and of the various advantages thereof can be realized by reference to the following detailed description in which reference is made to the accompanying drawings in which:

FIG. 1 is a perspective view of a driver and set screw assembly according to an embodiment of the present disclosure.

FIG. 2 is a perspective view of the driver of FIG. 1 without the set screw thereon.

FIG. 3 is a perspective view of the driver of FIG. 2 shown in conjunction with a drill.

FIG. 4 is an enlarged perspective view of a distal end of the driver of FIG. 2.

FIG. 5 is a cross-sectional view of the distal end of the driver of FIG. 4 taken along line A-A.

FIG. 6 is an exploded view of a distal end of the driver assembly of FIG. 3.

FIG. 7 is a perspective view of the set screw of FIG. 1.

FIG. 8 is an enlarged view of the distal end of the driver and the set screw of FIG. 1.

FIG. 9 is a cross-sectional view of the distal end of the driver coupled with the set screw of FIG. 1.

FIG. 10 is a cross-sectional view of the distal end of the driver coupled with the set screw of FIG. 1.

FIG. 11 is a perspective view of a driver according to another embodiment.

FIG. 12 is a cross-sectional view of a distal end of the driver of FIG. 11.

FIG. 13 is an exploded view of a driver and set screw according to another embodiment of the present disclosure.

FIG. 14 is a cross-sectional view of the driver of FIG. 13.

### DETAILED DESCRIPTION

In describing the preferred embodiments of the disclosure, specific terminology will be used for the sake of clarity. However, the disclosure is not intended to be limited to any specific terms used herein, and it is to be understood that each specific term includes all technical equivalents, which operate in a similar manner to accomplish a similar purpose. In the drawings and in the description which follows, the term “proximal” refers to the end of instrument, or a portion thereof, which is closest to the operator in use, while the term “distal” refers to the end of the instrument, or portion thereof, which is farthest from the operator in use. When referring to the human body, the term “proximal” means closer to the heart, the term “distal” means more distant form the heart, the term “anterior” means towards the front part of the body or the face and the term “posterior” means towards the back of the body. The term “medial” means toward the midline of the body and the term “lateral” means away from the midline of the body.

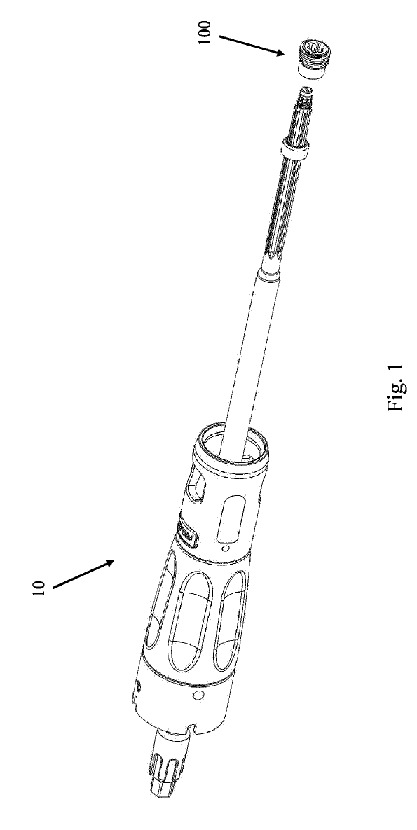

FIG. 1 is a perspective view of a driver 10 and set screw 100according to an embodiment of the present disclosure. Driver 10 is configured to receive set screw 100. While discussed largely in the context of use in implanting pedicle screws, it is contemplated that the drivers and sets screws disclosed herein could have applicability in other uses, surgical or otherwise. For instance, the driver and screw design could have applicability to the coupling of plates to implanted interbody devices. Moreover, certain aspects of the design could be implanted in different designs where appropriate. By way of non-limiting example, certain aspects of the set screw design could be incorporated in more traditional bone screws or the like and then such component could be useable with the same or similar driver shown in the figures.

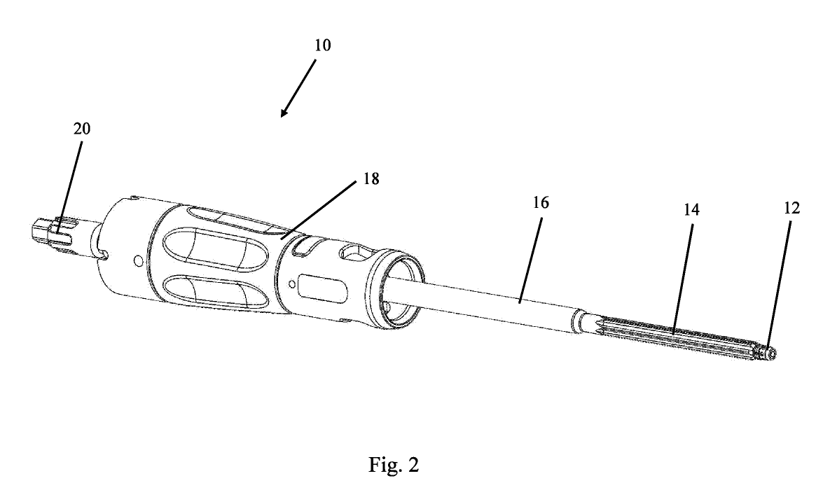

Referring to FIG. 2, driver 10 includes a retention tip 12, a driver shaft 14, an unthreaded shaft 16, a handle 18, and a coupling end 20. Retention tip 12 is fixedly, but rotatably, coupled to driver shaft 14, which is in turn, integrally formed with unthreaded shaft 16 and coupling end 20. Retention tip 12, driver shaft 14, and unthreaded shaft 16 are configured to rotate within handle 18 such that a user can hold handle 18 such that the remaining components can be rotated to implant set screw 100. Coupling end 20 is configured to engage with another component, such as, but not limited to, a driving tool (not shown) in the form of a ratchet/torque handle or a powered device such as a surgical drill. It should be appreciated that coupling end 20 can be of any shape or size to engage with a corresponding driving tool.

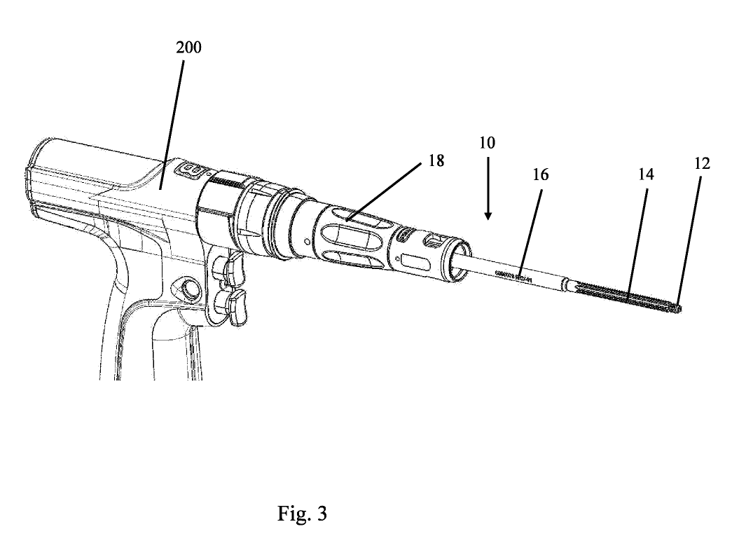

FIG. 3 depicts driver 10 coupled with a drill 200. Specifically, a distal end of driver 10, opposite retention tip 12 is configured to engage with an end of drill 200, such that actuation of drill 200 causes rotation of driver 10. It should be appreciated that drills other than drill 200 may be utilized with driver 10. Likewise, drivers other than driver 10 may be utilized with drill 200, such as, but not limited to, manual drivers.

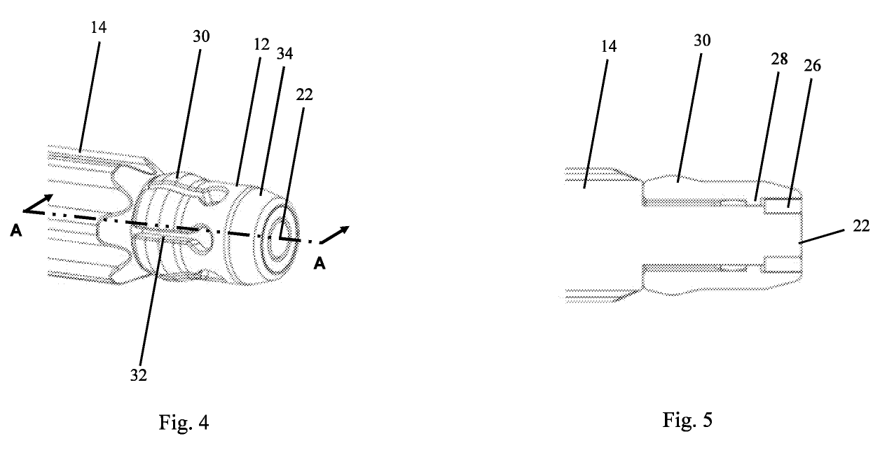

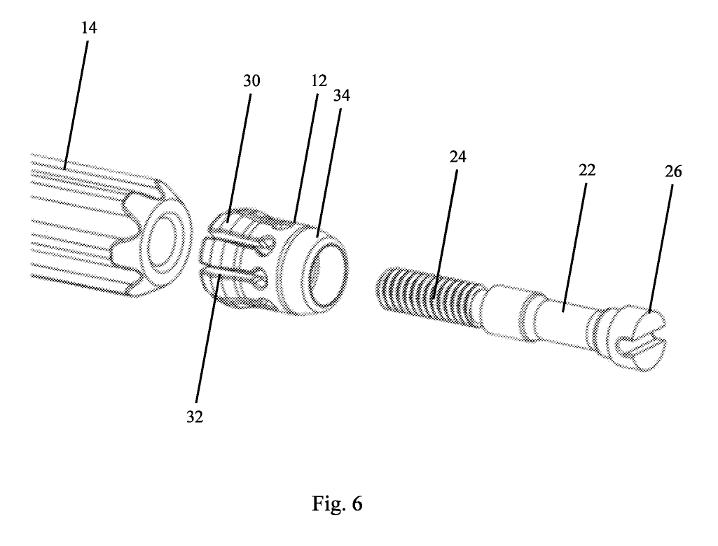

FIGS. 4-6 show the distal end of driver 10. Retention tip 12 is attached to driver shaft 14 via an attachment screw 22. As shown in FIGS. 5-6, attachment screw 22 includes a threaded portion 24 having a threaded surface configured to engage with the interior of driver shaft 14. When completely inserted, engagement of threaded portion 24 with the interior of driver shaft 14 causes a head 26 of attachment screw 22 to abut up against a shoulder 28 (FIG. 5) formed in the interior of retention tip 12 to secure the tip against the shaft. It should be appreciated that the engagement of the components still permits the rotation of retention tip 12 with respect to driver shaft 14. Indeed, screw 22 includes unthreaded portions that permit rotation even after attachment. It is to be understood that retention tip 12 could also be attached to the remainder of driver 10 in other manners, including but not limited to, welding or the like.

With continued reference to FIGS. 4-6, flexible portions 30 of retention tip 12 are configured to flex inward upon the application of a force, but are biased in an outward manner. A plurality of cutouts 32 are defined in retention tip 12 separating flexible portions 30. Specifically, each flexible portion 30 is between two cutouts 32 and each cutout 32 is between two flexible portions 30. A distal solid portion 34 of retention tip 12 has a smaller outer dimension than flexible portions 30. Moreover, distal solid portion 34 defines a smooth transition zone between an end of distal solid portion 34 and flexible portions 30. This permits the application of a set screw, such as set screw 100, in a smooth manner and one which results in the retention of the set screw to driver 10. Although a specific number of flexible portions 30 and cutouts 32 are shown in the drawings, it is contemplated that any number of such features could be included in retention tip 12. Moreover, it is contemplated that retention tip 12 may include other designs, such as, but not limited to, a single flexible portion 30 that is biased outwardly in a similar manner to that shown in the FIG. 4.

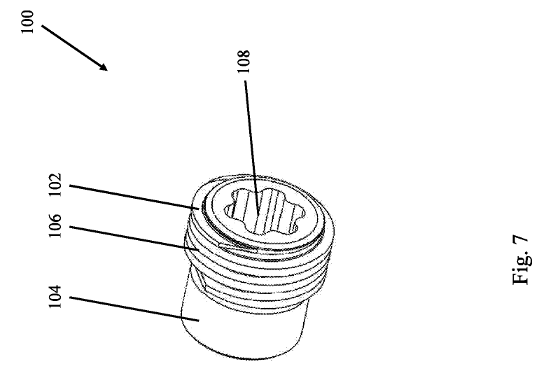

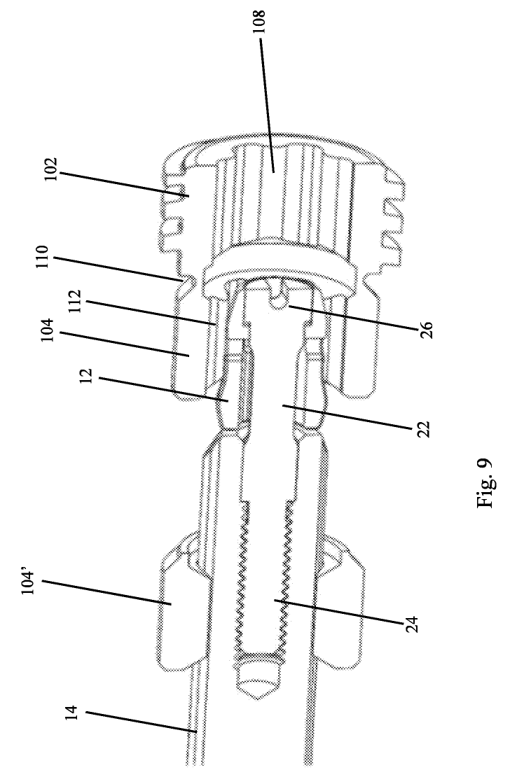

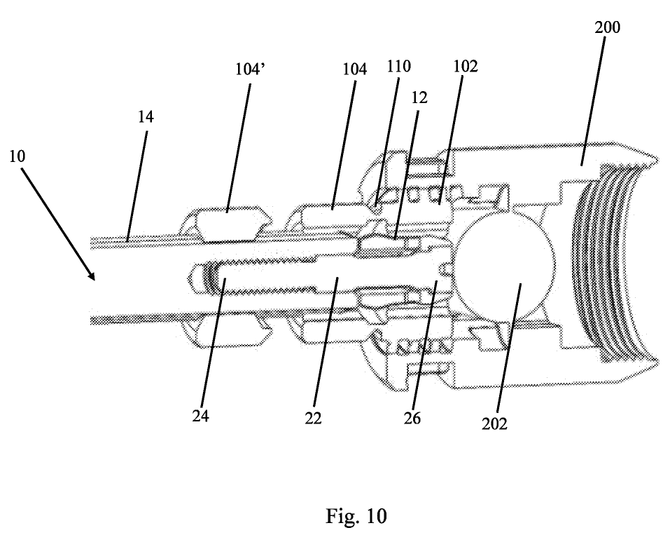

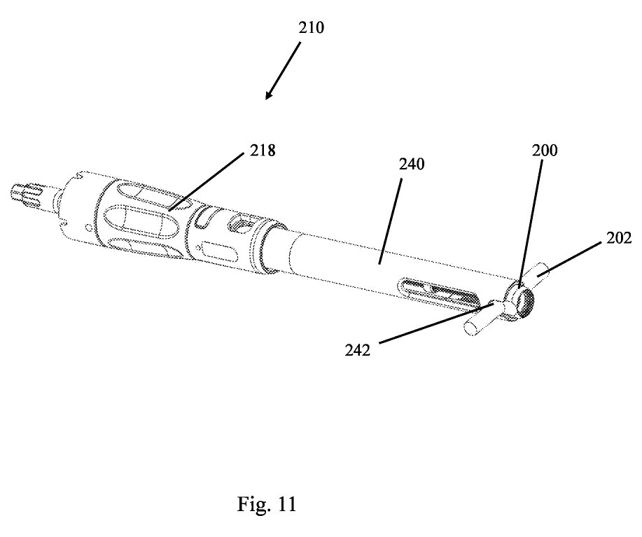

FIG. 7 shows a perspective view of set screw 100. Set screw 100 includes a screw portion 102 and a cap 104. Set screw 100 further includes an exterior thread 106 and an opening 108 configured to receive and engage with retention tip 12. Cap 104 is formed with screw portion 102 in a manner which permits cap 104 and screw portion 102 to separate upon an application of a maximum torque to cap 104. As shown in FIGS. 9-10, the attachment includes a weakened section 110 that is designed to fail upon reaching the maximum torque. Weakened section 110 is a necked-down area, but may include other shapes and forms (e.g., a coupling made of different material or an area lacking a solid structure) in other embodiments. As shown in FIG. 9, cap 104 includes an interior 112 that is configured to engage with the exterior of driver shaft 14 such that actuation of driver shaft 12 causes a rotational force to act upon cap 104. Cap 104 and driver shaft 14 define corresponding engaging surfaces. In an embodiment, the corresponding engaging surfaces are star-shaped surfaces. However, any type of corresponding engaging surfaces are contemplated, including different polygonal corresponding engaging surfaces.

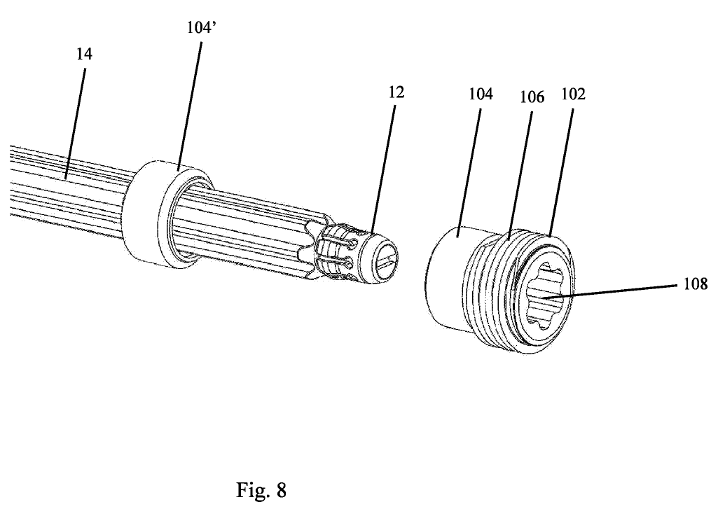

FIG. 8 shows a perspective view of the distal end of driver 10 and set screw 100. Of note, a cap 104’ from a prior implanted set screw is shown disposed on driver shaft 14. Indeed, the design of the shaft is such that already utilized caps can effectively be captured and stored on the shaft, thereby permitting a rapid implantation of multiple set screws. This is important in that most pedicle fusion screw procedures involve the implantation of several pedicle screws across multiple levels and on both side of the spine. The length of shaft 14 is such that a predetermined amount of removed caps can be retained thereon. This length can be modified depending upon the desired retention amount.

FIG. 9 depicts driver 10 and set screw 100 in a partial state of assembly. As shown, retention tip 12 has been introduced into interior 112 of cap 104, but not yet into opening 108 of screw portion 102. In this state, set screw 100 is not yet retained by driver 10. However, the solid portion 34 is shown beginning to enter opening 108, whereby the smooth transition will permit the ultimate introduction of flexible portions 30 as they flex inward.

FIG. 10 depicts driver 10 and set screw 100 in a full state of assembly and in relation to a coupling element 200 and spinal rod 202 of a pedicle screw construct. As shown, retention tip 12 is disposed within opening 108. This involves the compression of flexible portions 30 such that a friction fit is essentially created between opening 108 and retention tip 12. In this state, set screw 100 is, absent a force, held to driver 10. Driver shaft 14 also engages interior 112 of cap 104 such that a rotational force can be applied by driver shaft 14 to cap 104. Because retention tip 12 can freely rotate with respect to driver shaft 14, cap 104 in fact receives the entirety of this rotational force. Upon a complete insertion (including a reaching of a maximum torque/force) into coupling element 200, weakened section 110 effectively breaks, thereby separating cap 104 from screw portion 102. Again, the design of driver shaft 14 is such that the broken off cap can be retained, as demonstrated by retained cap 104’. The length of driver shaft 14 can be designed to capture a specific amount of broken off caps (e.g., ten caps), thereby permitting the rapid introduction and implantation of set screws to an entire pedicle screw construct.

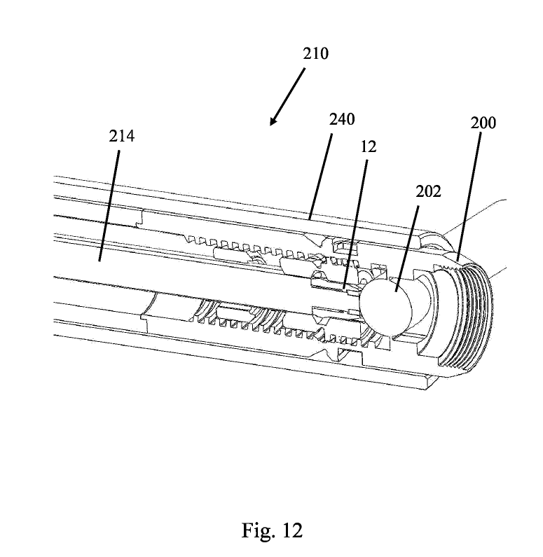

FIGS. 11 and 12 show a driver 210 according to another embodiment of the present disclosure. Because driver 210 shares similar elements with that of driver 10, like reference numerals will be utilized, but within the 200-series of numbers. For instance, driver 210 includes a handle 218. A difference between the embodiments is the inclusion of anti-torque tube 240 in driver 210. Tube 240 is fixed to the driver and slots 242 are keyed to spinal rod 202 such that an anti-torque function is provided. This prevents unwanted stress to the underlying vertebral bodies. As shown in FIG. 12, tube 240 is designed in certain embodiments to capture extended tabs of the pedicle screw.

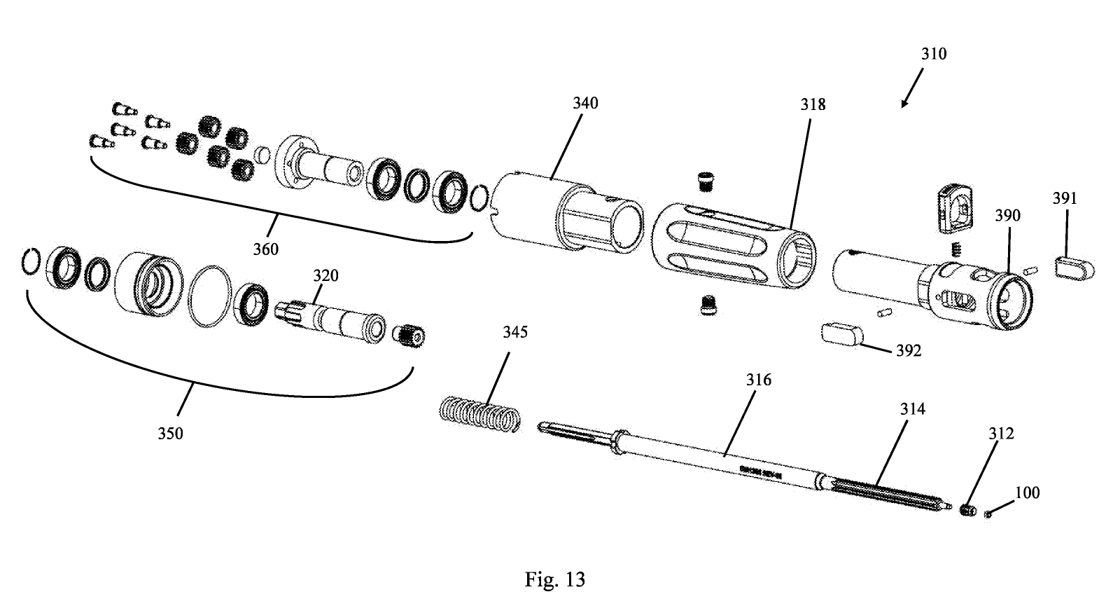

FIG. 13 shows an exploded view of a driver 310 and set screw 100 according to another embodiment of the present disclosure. Driver 310 shares similar elements to driver 10 and driver 210, and therefore like elements are referred to with similar numerals within 300-series of numbers. For example, driver 310 includes retention tip 312, driver shaft 314, unthreaded shaft 316, handle 318, and coupling end 320. Unlike driver 10 and driver 210, driver 310 includes a planetary gear system. Specifically, driver 310 includes a first subsection 350 of the planetary gear system and a second subsection 360 of the planetary gear system. Components of first subsection 350 and second subsection 360 are configured to couple with each other as well as other components in driver 310. As a non-limiting example, first subsection 350 and second subsection 360 are configured to engage with a portion the shaft of driver 310 opposite retention tip 312 (i.e., proximate unthreaded shaft 316). First subsection 350 and second subsection 360 include various components of a planetary gear system configured to change torque and rotational speed. For instance, the planetary gear system may reduce torque in exchange for higher rotational speed. As another example, the planetary gear system may increase torque in exchange for smaller rotational speeds. In this regard, driver 310 may be configured to provide greater torque capabilities.

With continued reference to FIG. 13, driver 310 further includes housing 340 configured to engage with handle 318 and receive at least some of the components of first subsection 350 and second subsection 360. Moreover, driver 310 includes housing 390 having interdigitating buttons 391, 392 coupled therein.

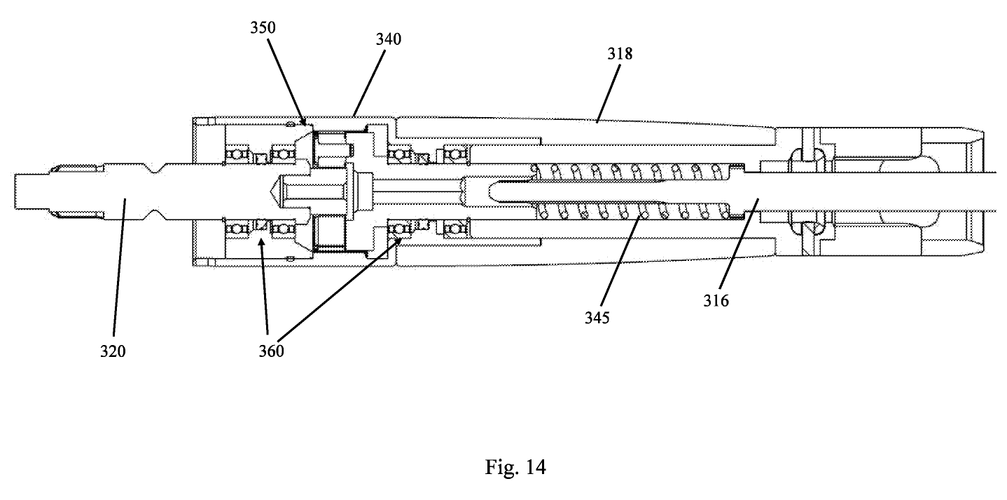

FIG. 14 shows a cross-sectional view of a portion of driver 310. As shown, first subsection 350 and second subsection 360 of the planetary gear system are positioned within driver 310 and are configured to actuate coupling end 320.

It is to be understood that the various components discussed herein can be constructed of any suitable material for use in the human body. For instance, it is contemplated to construct the various components of metallic materials, such as titanium and stainless steel. It is also contemplated to use various manufacturing methods to construct the components, including additive manufacturing. For instance, it may be particularly useful to form set screws in accordance with the present disclosure via an additive manufacturing process. Such may permit a more precise formation of weakened sections thereby providing for a more precise maximum torque limit before the caps separate from the screw portions.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A system comprising:
a set screw having a threaded portion and a cap; and
a driver having:
a shank configured to engage the cap, and
a retention tip including a flexible portion to retain the set screw to the driver,
wherein upon tightening of the set screw, the cap is removed from the threaded portion and retained by the shank.

2. The system of claim 1, wherein the cap is connected to the threaded portion by a weakened portion.

3. The system of claim 2, wherein the weakened portion is designed to break upon the application of a maximum torque to the cap.

4. The system of claim 1, wherein the retention tip is rotatable with respect to the shank.

5. The system of claim 4, wherein the retention tip is connected to the shank by a screw.

6. The system of claim 1, wherein the shank and cap have cooperating surfaces.

7. The system of claim 6, wherein the cooperating surfaces are star shaped.

8. The system of claim 1, wherein the driver further includes a handle, the shank being rotatable with respect to the handle.

9. The system of claim 1, further comprising a driving tool attached to the driver.

10. The system of claim 9, wherein the driving tool is a drill.

11. A method of attaching a set screw to an implant comprising:
engaging a retention tip a driver with a screw portion of the set screw;
coupling a shaft of the driver with a cap of the set screw;
imparting a force on the cap with the driver to screw the screw portion into the implant; and
after fully screwing the screw portion into the implant, removing the cap from the set screw and retaining the cap on the shaft.

12. The method of claim 11, wherein removing the cap from set screw includes imparting a maximum torque to the cap.

13. The method of claim 11, wherein retaining the cap on the shaft includes sliding the cap along the shaft.

14. The method of claim 11, wherein the steps are conducted for multiple set screws and multiple caps are retained on the shaft.

15. The method of claim 11, wherein the retention tip includes at least one flexible portion.

16. The method of claim 15, wherein the retention tip include a plurality of flexible portions.

17. The method of claim 11, further comprising holding a handle of the driver while imparting the force.

18. The method of claim 11, wherein the imparting step is conducted with a driving tool.

19. The method of claim 11, wherein the implant is a pedicle screw.

20. A system comprising:
a plurality of set screws each having a threaded portion and a cap; and
a driver having:
a shank configured to engage the caps, and
a retention tip including a flexible portion to retain the set screws to the driver,
wherein upon tightening of each set screw, the respective cap is removed from the respective threaded portion and retained by the shank,
wherein the shank includes a length designed to retain a predetermined number of removed caps.
